(19) United States
(12) Patent Application Publication
(10) Pub. No.: US 2005/0222633 A1
(43) Pub. Date: Oct. 6, 2005

(54) Implantable medical device with slot antenna formed therein

(75) Inventor: Kurt Olov Edvardsson, Linkoping (SE)

Correspondence Address: SCHIFF HARDIN LLP;Patent Department, 6600 Sears Tower, 233 South Wacker Drive, Chicago, IL, 60606, US

(73) Assignee: St. Jude Medical AB

(21) Appl. No.: 11/093,576

(22) Filed: Mar. 30, 2005

(30) Foreign Application Priority Data
Mar. 30, 2004 (SE) 0400849-6

(51) Int. Cl. A61N 1/375

(52) U.S. Cl. 607/36

(57) ABSTRACT

An implantable medical device has a housing containing electronic operating circuitry for the operation of the implantable medical device, and radio frequency circuitry for transmitting and/or receiving radio frequency signals. The implantable medical device has at least one surface portion made of an electrically conductive material. At least one slot is provided in the surface portion of the electrically conductive material and a slot feed is operatively interconnected between the radio frequency circuitry and the slot. The surface portion of the electrically conductive material and provided with the slot is adapted to operate as a transmitting and/or receiving antenna for the radio frequency signals.

### BACKGROUND OF THE INVENTION

1. Field of the Invention

The present invention relates to an implantable medical device.

2. Description of the Prior Art

In an implantable medical device, such as a cardiac pacemaker, an implantable cardioverter/defibrillator, or an insulin dispenser, telemetry is used, e.g. to change or modify operation characteristics of the implantable device or to readout data from the implantable medical device to monitor its function or to achieve information of the patient having the device implanted. Telemetry systems for implantable medical devices utilize radio-frequency energy to enable communication between the implantable device and an external programmer device.

Earlier telemetry systems used a rather low radio frequency, i.e. 8-300 kHz, as a carrier wavelength for communication between an antenna of the implantable device and an antenna of the external programmer device, which were inductively coupled to each other. Due to the very poor operating distance of this technique, the exterior antenna had to be located in close proximity to the implantable device, typically within a few inches. Further, the communication could ensue only at a low transmission data rate.

Recently, telemetry systems using a radio frequency data link operating at a much higher frequency, around 400 MHz, have been proposed, which enable two improvements to be made. Firstly, the antenna efficiency can be improved allowing extended range between the pacemaker and the external antenna. Secondly, the transmission data rate can be improved. Even higher frequencies can be used such as those within the ISM-band at 2400-2485.5 MHz.

U.S. Patent Application 2002/0095195 discloses an implantable medical device utilizing such far-field electromagnetic radiation to allow communication over a large distance. Two conductive halves of a housing for the implantable device act as a dipole antenna for radiating and receiving far-field radio frequency radiation modulated with telemetry data. An insulating header, in which therapy leads can be located, separates the conductive halves.

The aforementioned published application 2002/0095195 discloses a manner to utilize the limited space for the antenna function, but, nevertheless, there are several limitations of using an implantable medical device, in which two separated conductive halves of the housing act as a dipole antenna.

Firstly, a header made of dielectric material is disposed between the two conductive halves, thereby allowing external interfering radiation to enter the implantable device and interfere with signals transmitted within any of the two conductive halves or with signals transmitted across the dielectric header.

Secondly, in order to hermetically seal the two housing halves, a number of feed-throughs between them are needed since different electric circuitry is located in different housing halves to effectively use the available space.

Thirdly, the design of the implantable medical device does not allow for an optimum location of the therapy leads with respect to potential interference of the therapy signals by radio frequency signals fed to or received by the dipole antenna. The antenna type lacks a voltage node on its surface, where the electric field has a minimum, thereby affecting the therapy signals to a minimum extent.

Finally, the mechanical structure of this known implantable medical device seems not to be optimum: two housing portions of a conductive material have to produced, and to be fixed to and hermetically sealed against an intermediate piece of dielectric material. The manufacturing is further complicated by the need for a number of feed-throughs.

### SUMMARY OF THE INVENTION

It is an object of the present invention to provide an implantable medical device provided with an antenna that overcomes the above-mentioned problems.

It is a further object of the invention to provide such an implantable medical device that exhibits an overall improved antenna performance in comparison with implantable medical devices of the prior art. Higher antenna efficiency implies increased usable range for an exterior antenna.

A further object of the invention is to provide such an implantable medical device that is shielded from external radio frequency radiation in any direction.

A still further object of the invention is to provide such an implantable medical device that has a minimum number of feed-throughs in the housing thereof.

A yet further object of the invention is to provide such an implantable medical device that is provided with therapy lines protruding from the implantable medical device, where the therapy lines are located so as to be affected as little as possible by radiation transmitted and/or received by the antenna of the implantable medical device.

A still further object of the invention is to provide such an implantable medical device wherein the antenna is easy to manufacture to a low cost, easy to tune, and which antenna enables an efficient use of available space.

A yet further object of the invention is to provide such an implantable medical device that is reliable, and particularly mechanically durable.

The above objects are achieved according to the present invention by an implantable medical device having a housing containing electronic circuitry for the operation of therapy components of the implantable medical device and containing radio frequency circuitry for transmitting and/or receiving radio frequency signals, and the implantable medical device having at least a surface portion made of an electrically conductive material, preferably a metallic material. At least one slot is provided in the surface portion of the electrically conductive material and a slot feed is operatively interconnected between the radio frequency circuitry and the slot. The surface portion of the electrically conductive material provided with the slot is adapted to operate as a transmitting and/or receiving antenna for the radio frequency signals.

The surface portion of electrically conductive material may be a surface portion such as a portion of or the complete housing, or a portion of a dielectric header, which is metallized. Alternatively, the surface portion of the electrically conductive material is located internally within the housing. The surface portion may be made of sheet metal.

If the housing is an electrically conductive housing, the entire housing with the slot can be tailored to operate as an antenna for the radio frequency telemetry signals without any requirement of an insulating separation.

Further, the electric circuitry, the radio frequency circuitry, and the interconnection therebetween are enclosed by the electrically conductive housing to shield the electric circuitry, the radio frequency circuitry, and the interconnection from external radiation in any direction.

If therapy lines, such as those used in a cardiac pacemaker device, are provided, they are arranged to protrude from the housing close to a voltage node of an electromagnetic field that occurs when the surface portion of the electrically conductive material operates as an antenna for the radio frequency telemetry signals. Such provision provides for a minimum interference between the therapy lines and the antenna function.

Three different general principles regarding the shape and location of the slot are as follows. A slot that is open at one end thereof and which in the antenna literature often is referred to as a notch antenna can be used. A slot that is closed at both ends thereof and that is preferably provided with a backing cavity can be used. A slot that is closed at both ends thereof and that is formed as a through-hole to extend across the complete thickness of the implantable medical device can be used.

Several types of slot feeds are known in the antenna literature and some of them are suitable to be used in the present invention. Depending on the kind of slot, a conductor crossing the slot, an inductive coupling loop within the slot, a conductor crossing a portion of the slot and connected capacitively to the opposite edge of the slot, or a feeding including a kind of coaxial or wire feed-to-waveguide transition can be used. The latter feed preferably is used when the slot is provided with a backing cavity, where the backing cavity operates as a waveguide to feed the slot.

In the description below it will be understood that the antenna of the present invention is operable to transmit or receive radio frequency signals. Although terms may be used that suggests one specific signal direction, it will be appreciated that such a situation encompasses that signal direction and/or its reverse.

### BRIEF DESCRIPTION OF THE DRAWINGS

FIGS. 1a-b illustrate schematically, in a top view with a top cover removed, and in a side view, respectively, an implantable medical device according to a preferred embodiment of the present invention.

FIGS. 2a-b illustrate schematically, in a top view with a top cover removed, and in a side view, respectively, a further preferred embodiment of the implantable medical device according to the present invention.

FIGS. 3a-b illustrate schematically, in a top view with a top cover removed, and in a side view with a slot feeding removed, respectively, a still further preferred embodiment of the implantable medical device.

FIG. 4 illustrates schematically, in a top view with a top cover removed, a yet further preferred embodiment of the implantable medical device.

FIG. 5 illustrates schematically, in a side view with a slot feeding removed, a still further preferred embodiment of the implantable medical device.

FIG. 6 illustrates schematically, in a side view, an alternative embodiment of a slot feeding for use with the implantable medical device of FIG. 5.

FIG. 7 illustrates schematically, in a side view, another alternative embodiment of a slot feeding for use with the implantable medical device of FIG. 5.

FIGS. 8a-b illustrate schematically, in a top view with a top cover removed, and in a side view, respectively, a yet further preferred embodiment of the implantable medical device.

FIGS. 9-11 illustrate schematically, in top views, still further preferred embodiments of the implantable medical device.

Throughout the figures similar parts and components, and portions thereof, are denoted by identical reference numerals.

### DESCRIPTION OF THE PREFERRED EMBODIMENTS

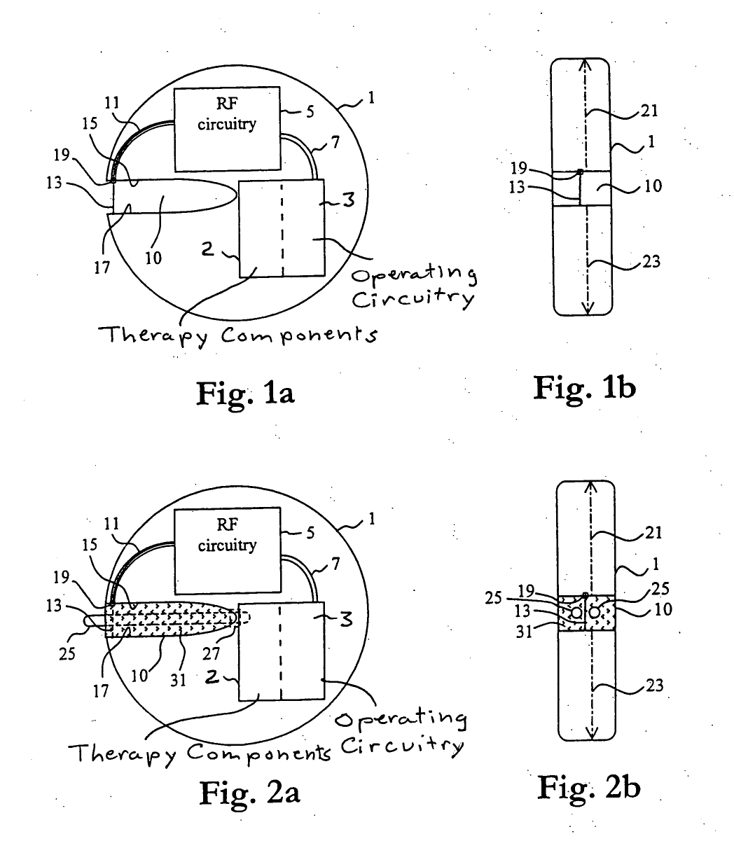

A first preferred embodiment of an implantable medical device of the present invention will be described with reference to FIGS. 1a-b. The implantable medical device e.g. may be a pacemaker, an insulin dispenser or other medical equipment including a telemetry link, preferably a high frequency telemetry link.

The implantable medical device has an electrically conductive hollow housing 1, therapy components 2 adapted to interact with a subject in whom the device is implanted, electronic operating circuitry 3 for the operation of the therapy components, and radio frequency circuitry 5 for transmitting and/or receiving radio telemetry frequency signals. The operating circuitry 3 and the radio frequency circuitry 5 are interconnected 7 and are arranged in the electrically conductive housing in a common space or in separate compartments, which may be shielded from each other.

According to the present invention the electrically conductive housing 1 is provided with a slot 10, and feed conductors 11, 13 are interconnected between the radio frequency circuitry 5 and edges 15, 17 of the slot 10. By means of such provisions, the electrically conductive housing can be adapted to operate as an antenna for the radio frequency telemetry signals.

In the FIGS. 1a-b embodiment the slot 10 is open in one end thereof to form what is usually referred to as a shunt or notch antenna in the antenna literature, see e.g. R. C. Johnson, Antenna Engineering Handbook, third edition, McGraw-Hill, 1993, pages 37-14-37-17, the content of which being hereby incorporated by reference. This kind of antenna has been used extensively on aircraft. The length of the slot should nominally be λ/4 to obtain resonance, where λ is the effective wavelength in the dielectric material in the slot. The length can be different if tuning components are included.

In FIGS. 1a-b the slot is open so that blood and surrounding tissue may fill out the slot. The dielectric constant of such matter may vary considerably, which naturally affect the resonance frequency or wavelength of the antenna. The dielectric material is not important for the radiation performance as such, but tunes the resonance to a lower frequency than the one indicated by the physical length. At 400 MHz and a dielectric constant of 1, a quarter of an effective wavelength to obtain resonance measures 18 cm, whereas at a dielectric constant of 65 the resonant slot length is 2.3 cm. Correspondingly, at 2.4 GHz a dielectric constant of 1 gives a resonant λ/4 length of 3.1 cm, whereas a dielectric constant of 65 gives a resonant λ/4 length of 0.39 cm. All these slot lengths but the largest are easily feasible to use in a cardiac pacemaker device, which today typically measures about 3-8 cm in diameter. Also, there is a wide possibility to use shorter slots by suitable impedance matching. The use of a λ/4 slot as being illustrated in FIGS. 1a-b is obviously preferred to keep the dimensions small, especially when a frequency around 400 MHz is used.

Preferably, the operating circuitry 3, the radio frequency circuitry 5, and the interconnection 7 are enclosed by the electrically conductive housing 1 so that the operating electric circuitry 3, the radio frequency circuitry 5, and the interconnection 7 are shielded from external radio frequency radiation in any direction. Hence, the electrically conductive housing 1 operates similarly to a Faraday cage to effectively hinder radio frequency radiation from entering the housing. With respect to the shielding functionality the conductive housing 1 may have openings of a size, which depends on the frequency of the radiation that shall be shielded. As the electrically conductive housing 1 is typically sealed, particularly hermetically sealed, sealed feed-throughs are required.

One of the feed conductors 13 is therefore provided with a hermetic feed-through 19 in the electrically conductive housing 1 so that the feed conductor 13 can protrude from one of the edges 15 of the electrically conductive housing 1, and extend across the slot 10 to be connected at the opposite edge 17 of the slot 10 to create an electric field within the slot 10 by flowing a feeding current in the feed conductor 13.

Preferably, the feed conductors are center 13 and shield 11 conductors of a coaxial cable, where the center conductor 13 is connected at the opposite edge 17 of the slot 10. The conductor, however, may be thicker than an ordinary center conductor of a coaxial cable in the slot 10.

The feed of the antenna may be implemented in any manner known in the art, e.g. using balanced feed or unbalanced feed, and using any kind of antenna tuning circuit or impedance matching network (not illustrated), which loads the antenna with a variable amount of inductance or capacitance to thereby adjust the effective electrical length of the antenna and match the antenna impedance to the impedance of the transmitter/receiver. In this manner, the reactance of the antenna may be tuned so that the antenna forms a resonant structure at the specified carrier frequency and efficiently transmits/receives far-field radio frequency radiation. Further, radiation-affecting components may be arranged across the slot 10. Capacitors, inductors, or active components may be interconnected between the edges 15, 17 of the slot 10. Capacitances may be implemented as small protrusions at the edges 15, 17 of the slot 10, whereas inductors may be implemented as narrow strips across slot 10.

The design of the impedance matching network and the slot 10 can be chosen in order to obtain suitable antenna performance in terms of radiation parameters such as resonance frequency, input impedance, bandwidth, radiation pattern, gain, polarization and near-field pattern.

Another way to control the feeding impedance is to move the location of the feed conductor 13 along the slot 10.

Preferably, the radiation parameters can be controlled to accommodate for different filling materials in the slot, which have different dielectric properties.

It will be appreciated that the feed conductor 13 does not necessarily have to cross the slot in its entire width. The feed conductor 13 may for instance make a loop in the slot and be connected to the same slot edge, at which it is fed through the electrically conductive housing 1.

The slot 10 in the FIGS. 1a-b is located substantially halfway between two oppositely located edges of the electrically conductive housing, i.e. so that extensions, of the electrically conductive housing 1 on either side of the slot in directions orthogonal to the slot 10, schematically indicated by arrows 21, 23, are of similar sizes. Such location of the slot provides for capabilities to obtain an optimum antenna performance. A location closer to the circumference of the housing 1 will gradually make the impedance matching more difficult.

FIGS. 2a-b illustrate a second embodiment of the implantable medical device wherein the device is provided with two output therapy lines 25 connected to the therapy components 2, with each of the therapy lines 25 being provided with a hermetic feed-through 27. The protrusions of the therapy lines 25 are only schematically indicated in FIGS. 2a-b and it shall be appreciated by the person skilled in the art that they may be longer. The therapy lines 25 may have different shapes and be different in number.

The therapy lines 25 preferably are located where they have as small influence on the antenna function as possible. This is useful both to avoid influence on the therapy function and to avoid the therapy lines to operate as antennas. In the antenna design procedure an electromagnetic field calculation is typically performed, and one result of such a calculation is the electric field around the implantable medical device, which will give hints for suitable locations. In order to thus minimize the interference the therapy lines 25 are located in the slot 10, preferably symmetrically in the slot, where the electric field has a minimum. An alternative location is at an opposite edge of the electrically conductive housing 1. The shape of the housing 1 may be less regular than what is illustrated in FIGS. 2a-b, but still a position on the circumference of the housing 1 can be found, where a local minimum of the electric field exists.

Further, the slot 10 is filled with a dielectric material 31, particularly a plastic, a glass or a ceramic material. This provides a well-defined dielectric constant of the material 31 in the slot, as well as providing good support for the therapy lines 25.

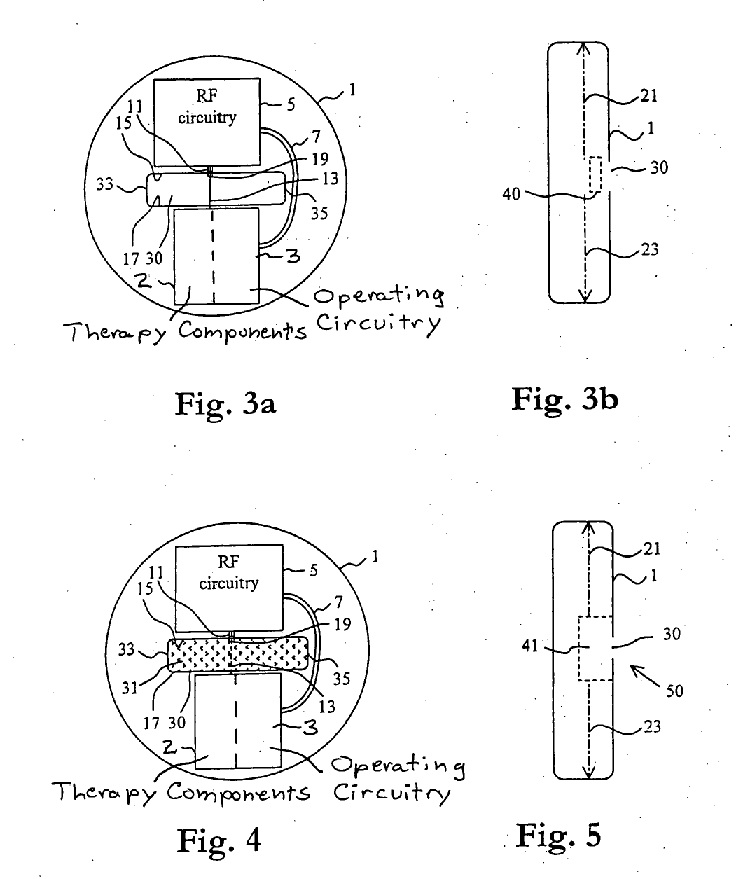

A third preferred embodiment of the implantable medical device of the present invention will next be described with reference to FIGS. 3a-b. The device comprises as above an electrically conductive housing 1, in which the operating circuitry 3, and the radio frequency circuitry 5 are interconnected. Feed conductors 11, 13 are interconnected between the radio frequency circuitry 5 and edges 15, 17 of the slot, which in this embodiment has different shape, and is therefore denoted by 30. The slot 30 is closed in both ends 33, 35 thereof and the antenna thus formed is commonly referred to as a slot or aperture antenna. A slot antenna is an elongated aperture in a conducting surface where one or more feeding elements generate an electric field over the elongated aperture.

The slot 30 preferably is half an effective wavelength (λ/2) long to achieve a resonant structure, but a shorter slot will radiate as well but with gradually larger impedance matching problems. A very short slot will also have a narrower instantaneous bandwidth. The shape is not critical for the antenna function and for instance rounded and wider ends, a so-called dumbbell shape, is frequently used to decrease the resonant length.

Below the slot 30 there is provided electric shielding or circuitry (schematically indicated at 40 in FIG. 3b) to prevent the slot from radiating inwards in the housing 1 to affect the circuitry therein in an adverse manner. The tissue of the body, which surrounds the implantable medical device, has typically a high electric constant, thereby reducing the length of the λ/2 slot. This also reduces the amount of radiation directed to the interior of the housing 1.

Obviously, there is a natural transition from the λ/2 slot geometry of FIGS. 3a-b to the λ/4 notch of FIGS. 1a-b. Provided that the λ/4 notch is made in a thin structure it can be seen as a λ/2 slot, which is bent over the edge of the structure.

FIG. 4 illustrates a fourth preferred embodiment of the implantable medical device, which is identical with the FIGS. 3a-b embodiment except of that the slot 30 is filled with dielectric material 31 having a known dielectric constant.

FIG. 5 illustrates a fifth preferred embodiment of the implantable medical device wherein the slot is what is referred to as a cavity-backed slot 50, and comprises a backing cavity 41 below the slot 30 instead of the shielding 40. In other respects this embodiment is identical with the FIGS. 3a-b embodiment.

The backing cavity 41 at the inside of the housing 1 is a rather large structure; it may measure λ/2 times λ/4−λ/2, where λ is the effective wavelength in the filling material of the backing cavity 41, if any. The antenna is a cavity resonator fed energized by a feed conductor connected across the slot (not illustrated), which radiates from the slot aperture. Further reference to cavity-backed slot antennas is given in R. C. Johnson, Antenna Engineering Handbook, third edition, McGraw-Hill, 1993, pages 8-7-8-9, the content of which is incorporated herein by reference. Depending on the dielectric constant of the filling material the depth of the cavity 41 will be different, but in the case the cavity should be deeper than the thickness of the implantable medical device, the backing cavity can be turned to be e.g. parallel with the surface of the housing 1, in which the thin slot is made.

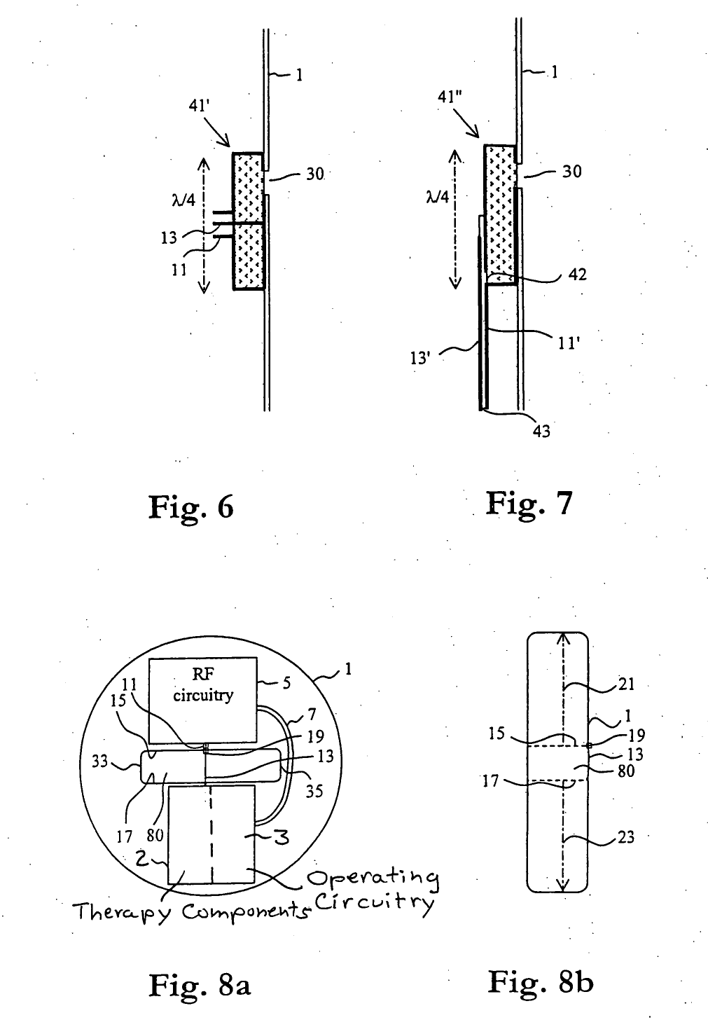

In FIG. 6 such a turned cavity 41′ for backing of the slot 30 is illustrated. The cavity can be filled with a ceramic material, which is partly metallized and welded or brazed to the electrically conductive housing to form a hermetic sealing. The feeding of the slot 30 can include a coaxial feed-to-waveguide transition. The center conductor 13 of the coaxial feeding is connected through a hole in the ceramic to an edge of the slot 30, whereas the shield conductor 11 is connected to the metallized ceramic. The ceramic should not be metallized in the slot 30. The length of the cavity 41′ is preferably about a quarter of an effective wavelength to obtain high impedance at the slot 30.

In FIG. 7 an alternative feed of the turned cavity-backed slot 50 is shown. A bottom end of the cavity, here denoted 41″, is provided with a second slot 42. A wire 13′, possibly on a printed circuit board 43, below the second slot 42 can be adapted to feed the second slot 42 to obtain a wire feed-to-waveguide transition. If the ceramic has a high dielectric constant the second slot 42 will be too short to radiate towards the electric circuitry in the housing 1. A ground connector 11′ or similar of the printed circuit board 43 is conveniently connected to the metallized portion of the ceramic-filled cavity 41″.

A sixth preferred embodiment of the implantable medical device is described with reference to FIGS. 8a-b. The device comprises as above an electrically conductive housing 1, in which the operating circuitry 3 and the radio frequency circuitry 5 are interconnected. Feed conductors 11, 13 are interconnected between the radio frequency circuitry 5 and edges 15, 17 of the slot, which in this embodiment is a through hole 80 through the complete thickness of the housing 1. This eliminates the need of a cavity.

It will be appreciated by those skilled in the art that in an alternative version of this embodiment, the through hole 80 is filled with dielectric material (not illustrated).

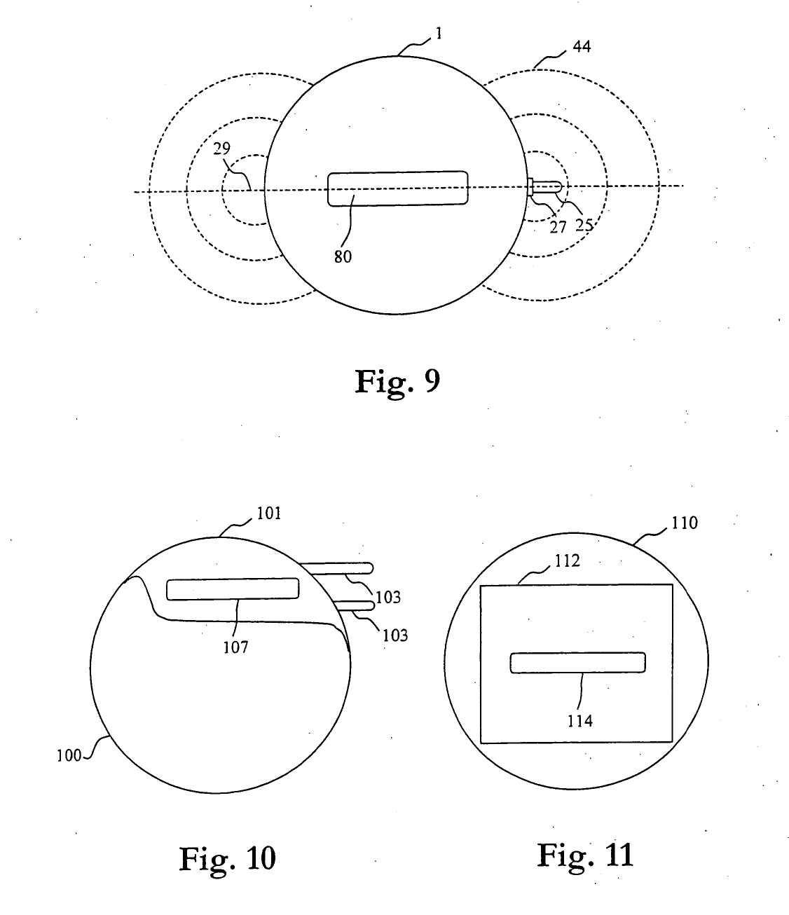

FIG. 9 illustrates a seventh preferred embodiment of the implantable medical device wherein the device is provided with a therapy line 25 protruding from the housing 1 via a hermetic feed-through 27. The therapy line 25 is connected to the electric circuitry of the implantable medical device (not illustrated in FIG. 9 for simplicity). The therapy line 25 protrudes from the housing 1 at a position along an extension line 29 of the slot 80. In other respects this embodiment is identical with the FIGS. 8a-b embodiment.

Generally, the therapy line 25 shall protrude from the housing 1 close to a voltage node of an electromagnetic field as obtained when the electrically conductive housing 1 operates as the antenna for the radio frequency signals. Such a voltage node can be found by calculating or measuring the electric field generated by the implantable medical device. In FIG. 9 equipotential surfaces of the electric field are denoted by 44.

FIG. 10 illustrates an eighth preferred embodiment of the implantable medical device. The hermetically sealed hollow housing 100 contains, as in the other preferred embodiments, operating circuitry, radio frequency circuitry, an interconnection between them, and antenna feed conductors (not explicitly illustrated).

The housing 100 may be of an electrically conductive material or of a dielectric material such as e.g. a ceramic. Further, a header 101 is mounted to the housing 100, wherein the header is of a dielectric material and is advantageously a molded component, e.g. an injection molded part.

The header 101 supports two therapy lines 103, which are connected to the electric circuitry in the housing 100 via hermetic feed-throughs in the wall of the housing 100.

According to the present invention a portion of or the complete surface of the header 101 is covered by metal, preferably in a process known as metallization, and a slot antenna 107 is formed in the metallized surface, e.g. by means of patterning and etching. Alternatively, the patterned metallization with the slot 107 is formed by some kind of direct printing technique.

Generally, any of the different slots described in this description may be used in this embodiment: a slot that is closed in both ends thereof and which is optionally provided with a backing cavity, a slot that is closed in both ends thereof and that is formed as a through-hole to extend across the complete thickness of the header 101, and a notch antenna. In the two latter cases, the header 101 has naturally to be formed to include a through hole or a notch structure, before the surface is metallized.

The feed conductors are advantageously connected to the antenna in any of the manners as described in connection with the descriptions of the other preferred embodiments of the invention. A hermetic feed-through for the feed conductors is provided in the wall of the housing 100.

If the housing 100 is of an electrically conductive material, the metallization of the header 101 is advantageously electrically connected to the housing 100.

FIG. 11 illustrates a ninth preferred embodiment of the implantable medical device. The housing or cover, here denoted by 110, is of a material, which is not electrically conductive, such as e.g. a ceramic or a plastic. The electric circuitry and the radio frequency may be arranged separately in shielded and/or hermetically sealed compartments within the housing 110, or together in a single compartment. If they are arranged in separate compartments hermetic feed throughs are needed for the connection between them. If the circuitry is arranged in a hermetically sealed environment, the housing 110 itself does not necessarily have to be hermetically sealed. The housing 110, for instance, may be molded.

The implantable medical device according to the present invention has a surface portion 112 made of an electrically conductive material, preferably a metal, wherein a slot 114 is provided in the surface portion 112 made of the electrically conductive material. A slot feed is operatively interconnected between the radio frequency circuitry and the slot 114, and the surface portion 112 made of the electrically conductive material provided with the slot 114 is adapted to operate as a transmitting and/or receiving antenna for radio frequency signals transmitted and/or received by the radio frequency circuitry. The slot feed may be similar to any of those illustrated in FIGS. 5-7.

Preferably, the surface portion 112 made of the electrically conductive material is an outer surface portion of the housing 110, and thus of the medical device. The conductive surface portion 112 with the slot 114 can be formed as a patterned metallization of e.g. one side of the housing 110.

Alternatively, the conductive surface portion 112 is an internal surface within the housing 110. It may for instance be an outer surface of a metallic wall compartment within the housing 110, or a metallization of the surface of a dielectric wall compartment.

Alternatively, the conductive surface portion 112 may be a piece of sheet metal, which is arranged within the housing 110.

In a further preferred embodiment of the invention, a large compartment or housing houses all components internal to the implantable medical device. It may be made of a metallic material or of a dielectric material, in which case a surface portion is metallized. The slot antenna of the invention is made in the wall of the compartment or housing, or in the metallized surface portion, and is connected as in any of the preferred embodiments above. The complete compartment or housing is then covered by a dielectric material, preferably a thin layer of the dielectric material, to obtain a medical device having an outer surface suitable to be implanted in a human being, and optionally to hermetically seal the medical device. Only therapy lines, if any, have to be capable of being connectable to the interior of the medical device.

It will be appreciated that the slots in the embodiments as described above does not have to be formed along a straight line, but may have any suitable elongated shape.

The slot-based antenna is used in the present invention i.e. as is it can be formed on a metallic structure, the shape of which may be determined by special requirements, and which should not be changed by the presence of the slot.

It will further be appreciated that the implantable medical device may be provided with two or more slots with separate feeding. Thus, such an implantable medical device may be adapted for transmitting and/or receiving radio frequency waves in at least two different frequency bands, e.g. both around 400 MHz and around 2.4 GHz.

The preferred embodiments described above are merely chosen to exemplify the present invention. Different features of different preferred embodiments may be combined to obtain yet further preferred embodiments of the invention.

Although modifications and changes may be suggested by those skilled in the art, it is the intention of the inventor to embody within the patent warranted hereon all changes and modifications as reasonably and properly come within the scope of his contribution to the art.

1. An implantable medical device comprising:
a housing;
medical therapy components contained in said housing;
electronic operating circuitry contained in said housing and connected to said therapy components for operating and controlling said therapy components;
radiofrequency circuitry contained in said housing and connected to said operating circuitry for transmitting and/or receiving radiofrequency signals associated with operation of said therapy components;
at least one surface portion at said housing comprised of electrically conductive material, said surface portion having a slot therein; and
a slot feed connected between said radiofrequency circuitry and said slot, said surface portion provided with said slot forming an antenna for said radiofrequency signals.

2. A device as claimed in claim 1 wherein said electrically conductive material is a metallic material.

3. A device as claimed in claim 1 wherein said surface portion comprises an outer surface portion of said housing.

4. A device as claimed in claim 1 wherein said housing comprises a header, and wherein said surface portion comprises at least a portion of said header.

5. A device as claimed in claim 4 comprising a therapy line connected to said therapy components and adapted for interaction with a subject to receive therapy, said therapy line protruding from said housing and being supported by said header.

6. A device as claimed in claim 5 wherein said housing is hermetically sealed and comprising a hermetic feed-through in said housing for said therapy line.

7. A device as claimed in claim 4 wherein said header comprises a dielectric part with a metallization thereon forming said surface portion.

8. A device as claimed in claim 7 wherein said slot is disposed in said metallization.

9. A device as claimed in claim 7 wherein said housing is comprised of electrically conductive material, and wherein said metallization is electrically connected to said electrically conductive material.

10. A device as claimed in claim 1 wherein said housing is comprised of electrically conductive material, and wherein said surface portion comprises said housing.

11. A device as claimed in claim 10 wherein said therapy components, said operating circuitry and said radiofrequency circuitry are enclosed by said electrically conductive housing to shield said therapy components, said operating circuitry and said radiofrequency circuitry from external radiation.

12. A device as claimed in claim 10 wherein said housing is hermetically sealed, and comprising a hermetic feed-through in said housing for said slot feed.

13. A device as claimed in claim 1 wherein said slot feed proceeds across said slot.

14. A device as claimed in claim 1 wherein said slot feed comprises an interior conductor and a shield of a coaxial cable.

15. A device as claimed in claim 1 wherein said slot has opposite closed ends.

16. A device as claimed in claim 15 wherein said slot comprises an interior radiation shielding.

17. A device as claimed in claim 15 wherein said slot is a cavity-backed slot.

18. A device as claimed in claim 15 wherein said slot is formed by a throughhole in said housing.

19. An implantable device as claimed in claim 1 wherein said slot has one open end.

20. A device as claimed in claim 19 comprising a therapy line connected to said therapy components, said slot comprising a hermetic feed-through for said therapy line.

21. A device as claimed in claim 1 wherein said slot is a cavity-backed slot.

22. A device as claimed in claim 21 wherein said slot feed comprises a coaxial feed-to-waveguide transition.

23. A device as claimed in claim 21 wherein said slot feed comprises a wire feed-to-waveguide transition.

24. A device as claimed in claim 1 wherein said housing is comprised of electrically conductive material and wherein said surface portion comprises said housing, and comprising a therapy line connected to said therapy components via a hermetic feed-through, said therapy line protruding from said housing at a location substantially aligned with said slot.

25. A device as claimed in claim 1 wherein said housing is comprised of electrically conductive material, and wherein said surface portion comprises said housing, and comprising a therapy line connected to said therapy components via a hermetic feed-through, said therapy line protruding from said housing at a location substantially at a voltage node of an electromagnetic field that occur when said electrically conductive housing operates as said antenna.

26. A device as claimed in claim 1 wherein said housing has opposite sides disposed substantially orthogonally to said slot, and wherein said slot is disposed substantially centrally between said opposite sides of said housing.

27. A device as claimed in claim 1 comprising dielectric material at least partially filling said slot.

28. A device as claimed in claim 27 wherein said dielectric material is a ceramic.
